SEQUENCE LISTING (2 sequences)

SEQ ID NO: 1 - DNA, 1584 bp, Phalaenopsis bellina
  CDS (1)..(1581)
atgctctcgaggctcagcggcgccgtttggagggacggagatgcggcagaggaggaagacgacaccgcttcatggataagatccacctcttgcaccgccgccatgggagaagtgagggacgacatcgccctcccctgtctcaaatctattctcgacgacgactggtacgtcgcagccacctcttccaacgccggtaacctcgccgggagctccttaaaccaggggttcgccggcgactcctccccgctcctttcgccgccggtagattcctcctgctcaccttcctccgtcttcacactcgatccctccgcccaccagtccttcttcccccacaaacccgcactttcttctttcctctctgctgtctgctccaacccgtttgattccggcttcgattttgtcccggagaacccgaccttcctcactccggcgaactccgcggcctcctcctccgtttttggtggtggaatgttgggtttccccaacgtgcagattaatagtcaagattttctgcctatgaccggaagctcttctttgttgaaccccatggccttcgattcgcttgaaaattcgccatttttgagcagagggaaggttttgaggcctctggagatctttccacaggtaggtgctcagcctaatctttttcagaaacgtgcggcggccgcactgcgccagaattcaagttctattggtggtgagaaaggtatgcgttttggggtttttggtatggaggctgcagggcagatgaggagctcttgggatggtgagctcgagaagcggaggagaatatatgatgaggatgagcttgatgatgccagcattgatggctctggtttaaattatgattctgaggatatctgtgaagttgcagagactgtaaagattgatgaagagaagaatgacggcgtaggagcagctgccggaggtggaggaggaggtaatgtttccattactaatagtagtcttaccggtgggggtggagagctgaagggtagaagaaaggggcttcctgctaagaatctgatggctgagaggcgtaggaggaagaagctcaatgacaggctttatatgctaagatctgttgttccaaagatcagtaagatggatagagcttccattctcggcgatgcaattgagtatttgaaagagcttttacagagaatcaatgaccttcacaatgaactcgagtcgacgaattcttccatgccggcatctagcacatcaagctttcaccccttaacgccgacgcccccaactcttccatgtcggatcaaggaggagctatgtccgagttcattgccgagcccaaatggtcaaccggcaagggtggaagtaagactcagagaaggcagggctgtaaacatacacatgttctgcgcccgtcggccgggtctcctcctctcgacgatgcgagctctcgaaggcctcggcctcgacatccagcaggccgtcataagctgcttcaacggcttcgctctcgatgtttttcgggccgagcaatctaaggatgggccaggtgttttaccagaagaaatcaaagaggttctgttgcattctgcaggcttccaaactgcactgtga

SEQ ID NO: 2 - protein, 527 aa, Phalaenopsis bellina
MLSRLSGAVWRDGDAAEEEDDTASWIRSTSCTAAMGEVRDDIALPCLKSILDDDWYVAATSSNAGNLAGSSLNQGFAGDSSPLLSPPVDSSCSPSSVFTLDPSAHQSFFPHKPALSSFLSAVCSNPFDSGFDFVPENPTFLTPANSAASSSVFGGGMLGFPNVQINSQDFLPMTGSSSLLNPMAFDSLENSPFLSRGKVLRPLEIFPQVGAQPNLFQKRAAAALRQNSSSIGGEKGMRFGVFGMEAAGQMRSSWDGELEKRRRIYDEDELDDASIDGSGLNYDSEDICEVAETVKIDEEKNDGVGAAAGGGGGGNVSITNSSLTGGGGELKGRRKGLPAKNLMAERRRRKKLNDRLYMLRSVVPKISKMDRASILGDAIEYLKELLQRINDLHNELESTNSSMPASSTSSFHPLTPTPPTLPCRIKEELCPSSLPSPNGQPARVEVRLREGRAVNIHMFCARRPGLLLSTMRALEGLGLDIQQAVISCFNGFALDVFRAEQSKDGPGVLPEEIKEVLLHSAGFQTAL

(19) United States
(12) Patent Application Publication
(10) Pub. No.: US 2020/0190529 A1
(43) Pub. Date: Jun. 18, 2020

(54) GENE, PROTEIN AND METHOD FOR IMPROVING AROMA PRODUCTION IN AN ORCHID

(71) Applicant: NATIONAL CHENG KUNG UNIVERSITY, TAINAN CITY (TW)

(72) Inventors: HONG-HWA CHEN, TAINAN CITY (TW); YU-CHEN CHUANG, TAINAN CITY (TW); WEN-CHIEH TSAI, TAINAN CITY (TW); YI-CHU HUNG, TAINAN CITY (TW); WEN-HUEI CHEN, TAINAN CITY (TW)

(21) Appl. No.: 16/222,278

(22) Filed: Dec. 17, 2018

(51) Int. Cl. C12N 15/82; C12N 15/74

(52) U.S. Cl. CPC C12N 15/8242; C12N 15/8205; C12N 15/743; C12N 15/8222

(57) ABSTRACT

The invention relates to a nucleic acid molecule that improves aroma production in an orchid, and a cell and a transgenic orchid comprising the nucleic acid molecule. The invention further relates to a polypeptide that improves aroma production in an orchid, and a method for improving the production of aroma in an orchid.

### BACKGROUND OF THE INVENTION

### 1. Field of the Invention

The invention relates to a gene for improving aroma production in a plant, and more particularly to a gene, protein and method for improving aroma production in an orchid.

### 2. Description of the Related Art

Terpenoids represent the largest group of plant floral volatiles (Tholl, 2015, Advances in Biochemical Engineering/Biotechnology 148, 63-106). They play important roles in attracting pollinators for successful reproduction (Blight et al., 1997, Journal of Chemical Ecology 23, 1715-1727; Byers et al., 2014, The Journal of Experimental Biology 217, 614-623) and in defense against pathogens and florivores (Junker et al., 2011, Journal of Chemical Ecology 37, 1323-1331; Huang et al., 2012, New Phytologist 193, 997-1008). Apart from their natural roles, terpenoids are widely used in the cosmetics and perfume industry and as food additives because of their unique aromas and flavors (Schwab et al., 2008, The Plant Journal 54, 712-732; Caputi and Aprea, 2011, Recent Patents on Food, Nutrition & Agriculture 3, 9-16).

The biosynthesis of terpene starts from the production of basic C5 units, isopentenyl diphosphate (IDP) and its isomer, dimethylallyl diphosphate (DMADP). Both C5 units are synthesized from the mevalonate (MVA) pathway in the cytosol or the methylerythritol phosphate (MEP) pathway in plastids. The C5 precursors derived from the MVA pathway are preferentially used for biosynthesis of sesquiterpenoids, and those generated by the MEP pathway are predominately used for monoterpenoids and diterpenoids. A group of enzymes called short-chain prenyltransferases are responsible for the successive condensation of IDP with DMADP to produce intermediates for terpene synthases (TPSs), including geranyl diphosphate synthase (GDPS) that produces geranyl diphosphate (GDP, C10) for monoterpenes, farnesyl diphosphate synthase that generates farnesyl diphosphate (FDP, C15) for sesquiterpenes, and geranylgeranyl diphosphate synthase that supplies geranylgeranyl diphosphate (C20) for diterpenes (Dudareva et al., 2004, Plant Physiology 135, 1893-1902).

Although our knowledge of the biosynthesis of floral terpenoids is increasing, little is known about its regulation. To date, the types of transcription factors (TFs) involved in terpene biosynthesis have been found to vary, and most studies on the regulation of terpene biosynthesis have been performed in vegetative tissues and fruit. In contrast, regulation of floral terpene biosynthesis has been less well studied. There has been only one study showing that AtMYC2 promotes inflorescence sesquiterpene production in Arabidopsis (Hong et al., 2012, The Plant Cell 24, 2635-2648).

### SUMMARY OF THE INVENTION

The present invention provides a nucleic acid molecule and a polypeptide that improve aroma production in a plant.

One subject of the invention is to provide an isolated nucleic acid molecule, which nucleic acid molecule is selected form the group consisting of:
(a) a nucleic acid molecule, PbbHLH4, comprising a nucleotide sequence of SEQ ID NO: 1;
(b) one or more nucleic acid molecules comprising degenerate sequences of the nucleotide sequences of SEQ ID NO: 1;
(c) a nucleic acid molecule encoding a polypeptide comprising an amino acid sequences of SEQ ID NO: 2;
(d) a nucleic acid molecule encoding a polypeptide comprising an amino acid sequences of SEQ ID NO: 2 with one or more of the amino acids have been modified by a deletion, an insertion and/or a substitution, wherein the polypeptide has ability of improving production of monoterpene and/or sesquiterpene in an orchid;
(e) a nucleic acid molecule encoding a polypeptide comprising an amino acid sequence with at least 85% similarity to SEQ ID NO: 2, wherein the polypeptide has ability of improving production of monoterpene and/or sesquiterpene in an orchid; and
(f) a nucleic acid molecule hybridizes under stringent hybridization conditions to any one of the nucleic acid molecules as defined in (a) to (e).

Another subject of the invention is to provide a vector comprising the nucleic acid molecule mentioned above.

Still another subject of the invention is to provide a cell comprising the isolated nucleic acid molecule mentioned above.

Still another subject of the invention is to provide a transgenic plant comprising the nucleic acid molecule mentioned above.

Still another subject of the invention is to provide a protein, which protein comprises a polypeptide selected from the group consisting of:
(a) a polypeptide, PbbHLH4 comprising an amino acid sequence of SEQ ID NO: 2;
(b) a polypeptide comprising an amino acid sequence of SEQ ID NO: 2 with one or more of the amino acids have been modified by a deletion, an insertion and/or a substitution, wherein the polypeptide has ability of improving production of monoterpene and/or sesquiterpene in an orchid; and
(c) a polypeptide comprising an amino acid sequence with at least 85% similarity to SEQ ID NO: 2, wherein the polypeptide has ability of improving production of monoterpene and/or sesquiterpene in an orchid.

Still another subject of the invention is to provide a method for improving production of aroma in a plant, which comprises increasing the expression of the protein mentioned above.

Still another subject of the invention is to provide a method for improving production of aroma in an orchid, which comprises transforming the orchid with the vector mentioned above.

### BRIEF DESCRIPTION OF THE DRAWINGS

FIG. 1. Expression patterns of GDPS and bHLH4 in orchid flowers. (A) Levels of emitted monoterpenes in scented P. bellina (from FIG. 1B) and P. Meidarland Bellina Age ‘ LM128’ (labeled as LM128), and scentless P. aphrodite (from FIG. 1B), P. mannii and P. javanica at the D+5 floral stage (5 d after anthesis). (B, C) Expression of GDPS (B) and bHLH4 (C) in D+5 flowers. Expression was normalized to that of Actin1. Data are means (±SE) from three replicates. Pairwise comparisons between groups were performed using Tukey's honestly significant difference test, and different letters indicate significant differences at α=0.05.

FIG. 2 illustrates scent compounds produced by the transient ectopic expression of PbbHLH4 in flowers of scentless P. aphrodite. Content of emitted terpenes from flowers with transient ectopic expression of GUS and PbbHLH4 was analysed at 5 d post-infiltration. GUS was used as a control. Data are means (±SE) from three infiltrations. (B) Expression level of bHLH4 in the flowers of PbbHLH4-expressing P. aphrodite as determined by quantitative real-time PCR. Expression was normalized to that of Actin1. Data are means (±SE) from three replicates.

### DETAILED DESCRIPTION OF THE INVENTION

One subject of the invention is to provide an isolated nucleic acid molecule, which nucleic acid molecule is selected form the group consisting of:
(a) a nucleic acid molecule, PbbHLH4, comprising a nucleotide sequence of SEQ ID NO: 1;
(b) one or more nucleic acid molecules comprising degenerate sequences of the nucleotide sequences of SEQ ID NO: 1;
(c) a nucleic acid molecule encoding a polypeptide comprising an amino acid sequences of SEQ ID NO: 2;
(d) a nucleic acid molecule encoding a polypeptide comprising an amino acid sequences of SEQ ID NO: 2 with one or more of the amino acids have been modified by a deletion, an insertion and/or a substitution, wherein the polypeptide has ability of improving production of monoterpene and/or sesquiterpene in an orchid;
(e) a nucleic acid molecule encoding a polypeptide comprising an amino acid sequence with at least 85% similarity to SEQ ID NO: 2, wherein the polypeptide has ability of improving production of monoterpene and/or sesquiterpene in an orchid; and
(f) a nucleic acid molecule hybridizes under stringent hybridization conditions to any one of the nucleic acid molecules as defined in (a) to (e).

As utilized in accordance with the present disclosure, the following terms, unless otherwise indicated, shall be understood to have the following meanings:

The term “isolated nucleic acid molecule” as used herein refers to a nucleic acid molecule that (1) is not associated with all or a portion of a nucleic acid molecule in which the isolated nucleic acid molecule is found in nature, (2) is linked to a nucleic acid molecule to which it is not linked in nature, or (3) does not occur in nature as part of a larger sequence. Preferably, the isolated nucleic acid molecule is a polynucleotide. Examples of the isolated nucleic acid molecule are genomic DNA, mRNA, cDNA, or synthetic origin or some combination thereof, which by virtue of its origin the isolated nucleic acid molecule. An additional nucleic acid that does not affect the function of the isolated nucleic acid molecule is preferably contained. For example, several numbers of nucleic acids are contained in the 5′ and 3′ untranscribed regions.

The term “polynucleotide” as referred to herein means single-stranded or double-stranded nucleic acid polymers of at least 10 bases in length. In certain embodiments, the nucleotides comprising the polynucleotide can be ribonucleotides or deoxyribonucleotides or a modified form of either type of nucleotide. Said modifications include base modifications such as bromuridine, ribose modifications such as arabinoside and 2′,3′-dideoxyribose and intemucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

In one preferred embodiment of the invention, the isolated nucleic acid molecule is (a) a nucleic acid molecule, PbbHLH4 comprising a nucleotide sequence of SEQ ID NO: 1, that is derived from Phalaenopsis bellina (Orchidaceae). The isolated nucleic acid molecule of the present invention encompasses alleles of the gene encoding a protein composed of the amino acid sequence represented by SEQ ID NO: 1. The complete cDNA of PbbHLH4 contains 1584 nucleotides and encodes an open reading frame of 1581 nucleotides corresponding to a predicted protein of 527 amino acids.

In one preferred embodiment of the invention, the isolated nucleic acid molecule is (b) one or more nucleic acid molecules comprising degenerate sequences of the nucleotide sequences of SEQ ID NO: 1. The isolated nucleic acid molecule of the present invention has a nucleotide sequence which is selected from the combinations of arbitrary codons corresponding to amino acid residues encoded by PbbHLH4. Selection of codons may be carried out by means of a customary method. For example, selection of codons may be carried out in consideration of the frequency of use of codons of the host.

In one preferred embodiment of the invention, the isolated nucleic acid molecule is (c) a nucleic acid molecule encoding a polypeptide, PbbHLH4 comprising an amino acid sequence of SEQ ID NO: 2. While not willing to be bound by any theory, it is believed that the PbbHLH4 protein transactivates geranyl diphosphate synthases (GDPSs) and/or terpene synthases (TPSs), thus has ability of improving production of monoterpene and/or sesquiterpene in an orchid.

The term “ability of improving production of monoterpene and/or sesquiterpene in an orchid” as referred to herein means that when expressing a gene or introducing a protein in an orchid, production of monoterpene and/or sesquiterpene in the orchid can be increased. The manner for assaying the such production may include collecting metabolites of the orchid, as described by Chuang et al. (2017, Botanical Studies 58, 50); and eluting the metabolites by hexane and identified by gas chromatography/high-resolution mass spectrometry (GC/HRMS), as described by Hsiao et al. (2006, BMC Plant Biology 6, 14; 2008, The Plant Journal 55, 719-733).

In one preferred embodiment of the invention, the isolated nucleic acid molecule is (d) a nucleic acid molecule encoding a polypeptide comprising an amino acid sequences of SEQ ID NO: 2 with one or more of the amino acids have been modified by a deletion, an insertion and/or a substitution, wherein the polypeptide has ability of improving production of monoterpene and/or sesquiterpene in an orchid. The site of the aforementioned amino acid sequence at which amino acids are deleted, substituted, or added is arbitrary, so long as a protein containing the resultant modified amino acid sequence exhibits ability of improving production of monoterpene and/or sesquiterpene in a plant. Similarly, the number of amino acids which are deleted, substituted, or added is arbitrary, so long as a protein composed of the resultant modified amino acid sequence exhibits ability of improving production of monoterpene and/or sesquiterpene in an orchid.

In one preferred embodiment of the invention, the isolated nucleic acid molecule is (e) a nucleic acid molecule encoding a polypeptide comprising an amino acid sequence with at least 85% similarity to SEQ ID NO: 2, wherein the polypeptide has ability of improving production of monoterpene and/or sesquiterpene in an orchid. As used herein, a polypeptide comprising an amino acid sequence with at least 85% similarity to a reference polypeptide (such as SEQ ID NO: 2) refers to a polypeptide that differ from the reference polypeptide by substitution, deletion or insertion. For example, one or more of an amino acid residue is substituted with another amino acid residue with similar properties (based on size, polarity, hydrophobicity, and the like). The amino acids may be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenylalanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

Furthermore, one or more codons encoding a cysteine residue affect a disulfide bond of a specific polypeptide, and thus a cysteine residue is deleted, and the residue can be substituted by another amino acid residue.

As compared with the case in which an amino acid residue is conservatively substituted on the basis of the aforementioned description, when an amino acid residue is arbitrarily substituted, characteristics of the resultant protein are slightly changed.

In one preferred embodiment of the invention, the nucleic acid molecule of (e) encodes a polypeptide comprising an amino acid sequence with at least 95%, 95% or 99% similarity to SEQ ID NO: 2, wherein the polypeptide has ability of improving production of monoterpene and/or sesquiterpene in an orchid. In one more preferred embodiment of the invention, the nucleic acid molecule of (e) encodes a polypeptide comprising an amino acid sequence with at least 99% similarity to SEQ ID NO: 2, wherein the polypeptide has ability of improving production of monoterpene and/or sesquiterpene in an orchid.

Modification (variation) of the aforementioned amino acid sequence may naturally occur through, for example, mutation or modification after translation. A naturally occurring gene (e.g., the PbbHLH4 gene of the present invention) may be modified artificially. The present invention encompasses all the modified genes having the aforementioned characteristics, regardless of causes or means for such modification and variation.

Examples of the aforementioned artificial means include genetic engineering methods such as site-specific mutagenesis; chemical synthesis methods such as a phosphate triester method and a phosphate amidite method; and combinations of the above methods. More specifically, synthesis of DNA may be carried out through chemical synthesis by means of a phosphoramidite method or a triester method. Alternatively, synthesis of DNA may be carried out by use of a commercially available automatic oligonucleotide synthesis apparatus. Double-stranded DNA fragments may be produced from single-stranded products which are chemically synthesized by annealing synthesized complementary strands under appropriate conditions or by adding complementary strands by use of an appropriate primer sequence and DNA polymerase.

In one preferred embodiment of the invention, the isolated nucleic acid molecule is (f) a nucleic acid molecule hybridizes under stringent hybridization conditions to any one of the nucleic acid molecules as defined in (a) to (e). The stringent conditions are not particularly limited, so long as the DNA fragment can be used as a primer or a probe. For example, the hybridization can be carried out under the condition as described above; i.e., in 0.2×SSC containing 0.1% SDS at 60° C., or in 0.1×SSC containing 0.1% SDS at 60° C.

Another subject of the invention is to provide a vector comprising the nucleic acid molecule mentioned above. In one preferred embodiment of the invention, the isolated nucleic acid molecule is contained in a vector. The vector is used for storing or producing the nucleic acid molecule, or introducing the nucleic acid molecule into a plant or a plant cell. Preferably, the vector is a shuttle vector. As used herein, the term “shuttle vector” refers to a vector, which can be manipulated and selected in both a plant and a convenient cloning host, such as a prokaryote. Such a shuttle vector may include a kanamycin resistance gene for selection in plant cells and an actinomycin resistance gene for selection in a bacterial host. Besides, the shuttle vector contains an origin of replication appropriate for the prokaryotic host used, and preferably at least one unique restriction site or a polylinker containing unique restriction sites to facilitate the construction.

In another aspect, the isolated nucleic acid molecule according to the invention is preferably driven by a promoter contained in the vector. More preferably, the promoter has ability to drive expression of a nucleic acid within at least one portion of the reproductive tissues in the recipient plant, such as the cauliflower mosaic virus 35S protein promoter, the α-1 and β-1 tubulin promoter, and the histone promoters. In one embodiment of the invention, the promoter is an inducible promoter comprising but not limited to heat-shock protein promoters and light-inducible promoters including the three chlorophyll a/b light harvesting protein promoters. The methods of vector construction are well known to those skilled in the art.

Another subject of the invention is to provide a cell comprising the isolated nucleic acid molecule mentioned above.

In one preferred embodiment of the invention, the cell is a prokaryotic cell, an eukaryotic cell, a plant cell, a monocot cell, an orchid cell, a Phalaenopsis spp. cell, and a cell derived from a protocorn-like body. The term “protocorn-like body” used herein refers to a tissue, which has a potential to differentiate and is ability for strong and rapid proliferation ability. Preferably, the nucleic acid molecule is introduced to the cell with transformation. As used herein, the term “transformation” refers to a process for changing the genetic material of a cell through introducing a nucleic acid molecule. Persons skilled in this art can conduct the transformation according to the disclosure of the invention and normal knowledge in molecular biology. For example, the vector may be introduced into a bacterial by heat shock process, or the vector is introduced into a plant cell by a gene gun.

Still another subject of the invention is to provide a transgenic plant comprising the nucleic acid molecule mentioned above. Preferably, the transgenic plant is an orchid; more preferably, the transgenic plant is a Phalaenopsis spp.

According to the invention, the plants to be transformed with the genes include orchid and orchid cells, preferably Phalaenopsis spp., which may be the wild type and an artificial mutant that produced by such as chemical modification, X-ray activated random mutagenesis, recombinant techniques, or somaclonal variation.

Preferably, the transgenic orchid comprising at least one cell transformed with the isolated nucleic acid molecule, which may be transformed by conventional methods known to persons skilled in the art.

In an embodiment of the invention, a transgenic plant can be obtained by regenerating a transformed plant cell with the genes of the invention that are capable of modifying the phenotype of the plant, wherein the cells of the transgenic plant all have the same genetic material. In another embodiment of the invention, a mosaic plant can be obtained by transforming some of cells in a plant, such as reproductive cells or tissues, with the genes of the invention, wherein only the transformed cells express the modified phenotype as compared to the parent plant.

In one preferred embodiment of the invention, a method for producing a transgenic orchid comprising the steps of:
(i) introducing the nucleic acid molecule according to the invention into an orchid cell to obtain an orchid transformed cell; and
(ii) regenerating the orchid transformed cell to obtain the transgenic orchid plant.

In one embodiment of the invention, a transgenic orchid plant may be produced through a protocorn-like body in vegetative planting or aspetic seed germination. After separating the cells in a protocom-like body, each can regenerate a new protocorn-like body and then a new plant. In step (a), the nucleic acid molecule is introduced into a protocorn-like body, and preferably through a gene gun. At this moment, the nucleic acid molecule is introduced into some cells in the protocorn-like body to form transformed cells, and some cells are not introduced with the molecule. The transformed cells can be selected with the marker of the vector. In step (b), the transformed cells are regenerated to transgenic plants. As used herein, the term “regeneration” refers to a growth process of a plant from a plant cell, a group of plant cells or a part of a plant. The method of regeneration is well known to persons skilled in this field. A transgenic orchid produced thereby is also provided in the invention.

Still another subject of the invention is to provide a protein, which protein comprises a polypeptide selected from the group consisting of:
(a) a polypeptide, PbbHLH4 comprising an amino acid sequence of SEQ ID NO: 2;
(b) a polypeptide comprising an amino acid sequence of SEQ ID NO: 2 with one or more of the amino acids have been modified by a deletion, an insertion and/or a substitution, wherein the polypeptide has ability of improving production of monoterpene and/or sesquiterpene in an orchid; and
(c) a polypeptide comprising an amino acid sequence with at least 85% similarity to SEQ ID NO: 2, wherein the polypeptide has ability of improving production of monoterpene and/or sesquiterpene in an orchid.

In one preferred embodiment of the invention, the protein has an amino acid sequence of SEQ ID NO: 2.

Still another subject of the invention is to provide a method for improving the production of aroma in an orchid, which comprises increasing the expression of the protein mentioned above.

Still another subject of the invention is to provide a method for improving production of aroma in an orchid, which comprises transforming the orchid with the vector mentioned above.

In a preferred embodiment of the invention, the orchid may be infiltrated by any proper host containing the vector mentioned above, such as Agrobacterium tumefaciens.

In one embodiment of the invention, the expression of the proteins can be changed by increasing the ploid of the nucleic acid molecule encoding the proteins in at least one cell of the plant. In a preferred embodiment of the invention, a gene gun is used to introduce the nucleic acid molecule into the cell for changing the expression of the protein.

Preferably, the aroma comprises a monoterpene and a sesquiterpene.

The following Examples are given for the purpose of illustration only and are not intended to limit the scope of the present invention.

### Example

Materials and Methods

Plant Material and Growth Conditions

Five Phalaenopsis orchids including two scented and three scentless ones that are commonly utilized as breeding parents were used in this study. The two scented orchids, P. bellina and P. Meidarland Bellina Age ‘LM128’, were purchased from Ming-Hui Orchids Nursery (Yunlin, Taiwan) and Meidarland Orchids (Tainan, Taiwan), respectively. The three scentless orchids were P. javanica (from Mi-Tuo Orchids, Kaohsiung, Taiwan), P. mannii (from Ji An Guang Feng, Hualien, Taiwan), and P. aphrodite subsp. formosana. The scented orchid (P. bellina) has a much bigger genome than the scentless P. aphrodite (15.03 pg/2C versus 2.80 pg/2C) (Lin et al., 2001, Journal of the American Society for Horticultural Science 126, 195-199). All plants were kept in a greenhouse at the National Cheng Kung University (NCKU, Tainan, Taiwan) under natural conditions.

Chromatographic Analysis of Floral Volatiles

The floral metabolites of the Phalaenopsis orchids were collected on day 5 post-anthesis (D+5) for 6 h (from 10.00 h to 16.00 h) as this represents their maximum emission interval (Chuang et al., 2017, Botanical Studies 58, 50). The flowers on the plants were placed in a scent-extracting apparatus as described by Chuang et al. (2017, Botanical Studies 58, 50). To analyse the scent composition, metabolites were sampled from a single flower, with three biological replicates. As a negative control, metabolites originating from the scent-extracting apparatus were analysed for background. The volatiles collected were eluted by hexane and identified by gas chromatography/high-resolution mass spectrometry (GC/HRMS) at the NCKU Instrument Center, as described by Hsiao et al. (2006, BMC Plant Biology 6, 14; 2008, The Plant Journal 55, 719-733).

Transcriptome Construction, Assembly, and Annotation

Transcriptomes were constructed for four floral stages of P. bellina: anthesis day (D0), D+3, D+5, and D+7. Total RNA was extracted from the entire flowers with duplicate biological repeats as described previously (Hsiao et al., 2006, BMC Plant Biology 6, 14; 2008, The Plant Journal 55, 719-733). Quality control of RNA was performed using the RNA 6000 Nano Assay supplied with the Agilent 2100 bioanalyser. Preparation of four cDNA libraries and subsequent sequencing was carried out using an Illumina HiSeq 2000 at the Beijing Genomics Institute. De novo assembly for whole-transcriptome construction was carried out using the clean reads from the four libraries by using Trinity (release-20130225) (Grabherr et al., 2011, Nature Biotechnology 29, 644), and the resulting sequences were the unigenes. The expression level of each unigene in each sample was calculated as fragments per kilobase of transcript per million mapped reads (FPKM) based on the number of fragments uniquely aligned to the unigene in each library.

To gain insight into the function of the unigenes, the non-redundant (Nr) protein database at NCBI (https://www.ncbi.nlm.nih.gov/) was used to annotate them by using BLASTx with an E-value cut-off of 1.0×10⁻⁵. The floral transcriptome of P. aphrodite and its annotation were downloaded from Orchidstra (Su et al., 2011, Plant & Cell Physiology 52, 1501-1514; 2013a, Plant & Cell Physiology 54, ell; 2013b, PLoS ONE 8, e80462), upgraded to Orchidstra 2.0 (http://orchidstra2.abrc.sinica.edu.tw/orchidstra2/index.php), in which the relative expression of unigenes is derived from microarray analyses at both the floral bud and full-blossom stages. Since two different methods were used to generate the Phalaenopsis transcriptome data, the most significant results were validated experimentally by quantitative real-time PCR.

Transient Ectopic Expression of TFs in Orchids

Plasmids were constructed as described previously (Hsu et al., 2015, Plant Physiology 168, 175-191). The coding sequences of PbbHLH4 was amplified from full-bloom flowers of P. bellina using genespecific primers and transferred to the vector p1304NhXb under a duplicated CaMV 35S promoter. Agrobacterium tumefaciens EHA105 carrying the resulting clones was infiltrated into scentless flowers of P. Aphrodite on the day of anthesis (D0) with three replicates as described previously (Hsu et al., 2015, Plant Physiology 168, 175-191). The promoter containing GUS only was used as the negative control.

Identification of Transcription Factors (TFs) Related to Terpene Biosynthesis

To isolate TFs and regulators, 28 193 proteins in the PlnTFDB database (ver. 3.0; Perez-Rodriguez et al., 2010, Nucleic Acids Research 38, D822-D827) were downloaded as queries to search the P. bellina transcriptome by using TBLASTn with an E-value cutoff of 1.0×10⁻⁵⁰. The resulting sequences were then classified by using iTAK (Zheng et al., 2016, Molecular Plant 9, 1667-1670). Among them, 335 genes annotated as bHLH, bZIP, ERF, NAC, MYB, and WRKY were isolated, and 165 genes with FPKM>1 were imported into the Short Time-series Expression Miner (STEM) software (Ernst and Bar-Joseph, 2006) for classification of their expression profiles. For STEM analysis, the temporal expression profiles were transformed to start at 0 by subtracting the FPKM levels of the four floral stages (D0, D+3, D+5, and D+7) by the value for the first stage. The STEM Clustering Method provided by the software was used to cluster the factors into 10 profiles according to their expression patterns.

Quantitative Real-Time PCR

Total RNA was extracted as described previously (Hsiao et al., 2006, BMC Plant Biology 6, 14; 2008, The Plant Journal 55, 719-733). For P. Meidarland Bellina Age ‘LM128’, P. aphrodite, P. javanica, and P. mannii, total RNA was extracted at the floral D+5 stage. For P. bellina total RNA was extracted at five floral stages: D−1, D0, D+3, D+5, and D+7. After removal of DNA contamination by DNase (NEB, UK), the RNA samples were reverse-transcribed to cDNA using SuperScript III (ThermoFisher Scientific). Primers were designed to detect transcripts of P. bellina and P. aphrodite simultaneously based on the corresponding transcripts in the two orchid transcriptomes. Quantitative real-time PCR was carried out using a StepOnePlus Quantitative Real-Time PCR System and a SYBR Green kit (Applied Biosystems) as described previously (Hsu et al., 2015, Plant Physiology 168, 175-191). The expression of all genes was normalized to the reference gene, PbActin1. Three biological replicates were used, and pairwise comparisons between groups were performed using Tukey's honestly significant difference test at α=0.05.

Results

GDPS May have Accounted for the Monoterpene Biosynthesis in the Orchids

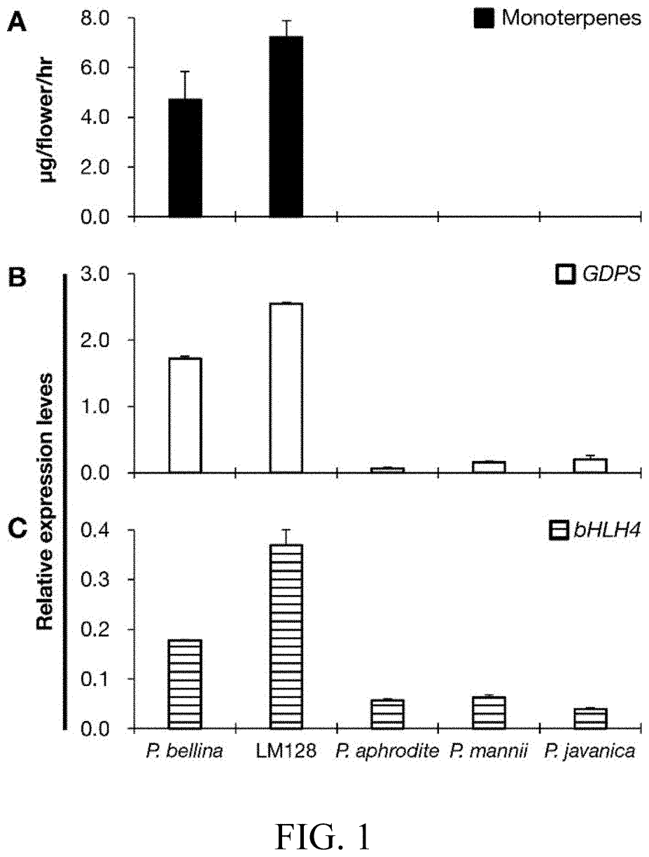

The levels of monoterpenes emitted from a single flower of the two species P. bellina, P. Meidarland Bellina Age ‘LM128’, P. javanica, P. mannii, and P. aphrodite subsp. formosana were detected (FIG. 1A). The expression levels of GDPS in them were also examined (FIG. 1B).

We detected significant differential expression of GDPS between the scented orchid (e.g., P. bellina) and the scentless orchid (e.g., P. aphrodite). The differential expression patterns of GDPS were concomitant with monoterpene production, which implied its strong association with the scented orchid phenotype. GDPS provides the precursors for further monoterpene biosynthesis, and the enhanced expression of GDPS may have accounted for the monoterpene biosynthesis in the orchids. Hence, the absence of elevated GDPS expression in the scentless P. aphrodite might be responsible for the lack of monoterpene accumulation.

Correlation Analysis to Identify Transcription Factors (TFs) Associated with PbGDPS Expression

Several types of TFs for terpene biosynthesis have been detected in various plant species, including bHLH, bZIP, ERF, NAC, MYB, and WRKY. A total of 335 TFs categorized in these types were identified in the P. bellina transcriptome by a BLAST search of PlnTFDB (Perez-Rodriguez et al., 2010, Nucleic Acids Research 38, D822-D827) and classified by iTAK (Zheng et al., 2016, Molecular Plant 9, 1667-1670). Because PbGDPS was highly expressed in the P. bellina transcriptome (FPKM>30), we selected 165 TF genes showing FPKM values >1 for further analysis. We applied two criteria to identify candidate TFs regulating GDPS for monoterpene biosynthesis: (1) the TFs had to appear either prior to or concurrent with the expression pattern of PbGDPS during flower development, and (2) the expression of the TFs had to be up-regulated in P. bellina but down-regulated in P. aphrodite. Among them, we found a transcription factor, namely PbbHLH4, showed enhanced differential expression between the two transcriptomes.

Expression of bHLH4 is Concomitant with Monoterpene Biosynthesis in Phalaenopsis Orchids

The expression levels of bHLH4 were further examined in the scented and scentless orchids (FIG. 1C). The bHLH4 genes were highly up-regulated in the commercial scented cultivar P. Meidarland Bellina Age ‘LM128’, an offspring of P. bellina that emitted high levels of monoterpenoids. In contrast, the expression levels of both genes were down-regulated in two native scentless orchids, P. javanica and P. mannii. The differential expression patterns of bHLH4 were concomitant with monoterpene production, which implied their strong association with the scented orchid phenotype.

Transient Ectopic Expression of PbbHLH4 in the Scentless Orchid

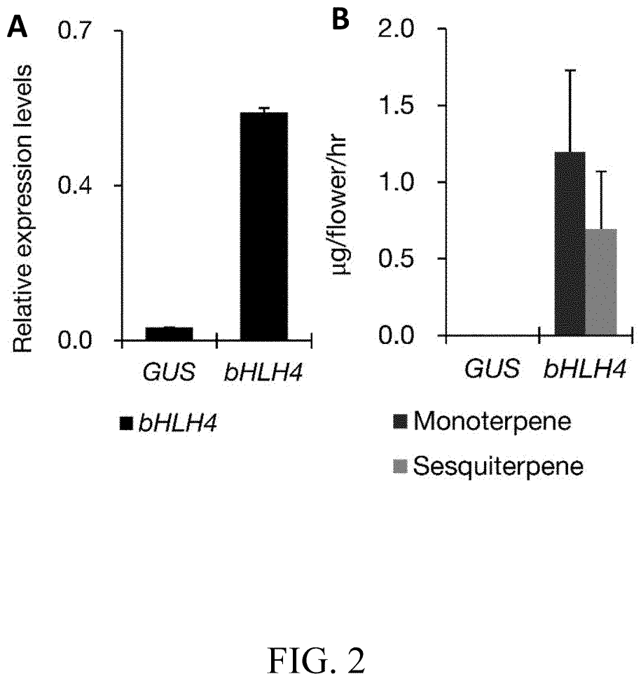

We hypothesized that ectopic expression of PbbHLH4 in P. aphrodite might induce a scented phenotype. Efficient stable transformation systems for Phalaenopsis orchids are lacking, with those available having low transformation efficiency as well as long regeneration times. Therefore, to test our hypothesis we used an efficient and rapid transient expression assay by utilizing Agrobacterium infiltration in the perianths of P. aphrodite (Hsu et al., 2015, Plant Physiology 168, 175-191). The coding sequences of PbbHLH4 was constructed under the control of double CaMV 35S promoters and introduced into P. aphrodite flowers on the day of anthesis (D0). The expression level of bHLH4 in the flowers of PbbHLH4-expressing P. Aphrodite was determined by quantitative real-time PCR (FIG. 2A). The floral scents emitted from these flowers were measured and identified using GC-MS analysis at 4 d post-infiltration. We detected a profound enhancement (˜950-fold) in a group of monoterpenoid and sesquiterpenoid emissions in PbbHLH4-expressing P. aphrodite flowers as compared with the GUS controls (1.89 μg versus 0.002 μg h-1 per flower) (FIG. 2B). The monoterpenoids were dominated by terpineol derivatives, such as α-terpineol (the major component), β-terpineol, γ-terpineol, 1-terpineol, and terpinolene, together with a trace amounts of 1,4-cineole, limonene, fenchone, and camphor. Two sesquiterpenoids, α-cedrene and β-cedrene, were also detected in the infiltrated flowers.

While embodiments of the present invention have been illustrated and described, various modifications and improvements can be made by persons skilled in the art. The present invention is not limited to the particular forms as illustrated, and that all the modifications not departing from the spirit and scope of the present invention are within the scope as defined in the appended claims.

What is claimed is:

1. An isolated nucleic acid molecule, which nucleic acid molecule is selected from the group consisting of:
(a) a nucleic acid molecule, PbbHLH4, comprising a nucleotide sequence of SEQ ID NO: 1;
(b) one or more nucleic acid molecules comprising degenerate sequences of the nucleotide sequences of SEQ ID NO: 1;
(c) a nucleic acid molecule encoding a polypeptide comprising an amino acid sequences of SEQ ID NO: 2;
(d) a nucleic acid molecule encoding a polypeptide comprising an amino acid sequences of SEQ ID NO: 2 with one or more of the amino acids have been modified by a deletion, an insertion and/or a substitution, wherein the polypeptide has ability of improving production of monoterpene and/or sesquiterpene in an orchid;
(e) a nucleic acid molecule encoding a polypeptide comprising an amino acid sequence with at least 85% similarity to SEQ ID NO: 2, wherein the polypeptide has ability of improving production of monoterpene and/or sesquiterpene in an orchid; and
(f) a nucleic acid molecule hybridizes under stringent hybridization conditions to any one of the nucleic acid molecules as defined in (a) to (e).

2. The isolated nucleic acid molecule according to claim 1, wherein the nucleic acid molecule of (e) encodes a polypeptide comprising an amino acid sequence with at least 95% similarity to SEQ ID NO: 2, wherein the polypeptide has ability of improving production of monoterpene and/or sesquiterpene in an orchid.

3. A vector comprising the nucleic acid molecule according to claim 1.

4. The vector according to claim 3, which is a shuttle vector that is capable of expressing the nucleic acid molecule in a plant.

5. The vector according to claim 3, which comprises an inducible promoter.

6. A cell comprising the isolated nucleic acid molecule according to claim 1.

7. The cell according to claim 6, which is a prokaryotic cell.

8. A transgenic orchid comprising the nucleic acid molecule according to claim 1.

9. The transgenic orchid according to claim 8, which is a Phalaenopsis spp.

10. A protein, which protein comprises a polypeptide selected from the group consisting of:
(a) a polypeptide, PbbHLH4 comprising an amino acid sequence of SEQ ID NO: 2;
(b) a polypeptide comprising an amino acid sequence of SEQ ID NO: 2 with one or more of the amino acids have been modified by a deletion, an insertion and/or a substitution, wherein the polypeptide has ability of improving production of monoterpene and/or sesquiterpene in an orchid; and
(c) a polypeptide comprising an amino acid sequence with at least 85% similarity to SEQ ID NO: 2, wherein the polypeptide has ability of improving production of monoterpene and/or sesquiterpene in an orchid.

11. The protein according to claim 10, wherein the polypeptide of (c) encodes a polypeptide comprising an amino acid sequence with at least 95% similarity to SEQ ID NO: 2, wherein the polypeptide has ability of improving production of monoterpene and/or sesquiterpene in an orchid.

12. A method for improving the production of aroma in an orchid, which comprises increasing the expression of the protein according to claim 10.

13. The method according to claim 12, wherein increasing the expression of the protein is by increasing the ploid of a nucleic acid molecule encoding the protein in at least one cell of the orchid.

14. The method according to claim 12, wherein the aroma comprises a monoterpene and a sesquiterpene.

15. A method for improving the production of aroma in an orchid, which comprises increasing the expression of a protein, which protein comprises a polypeptide selected from the group consisting of:
(a) a polypeptide, PbbHLH4 comprising an amino acid sequence of SEQ ID NO: 2;
(b) a polypeptide comprising an amino acid sequence of SEQ ID NO: 2 with one or more of the amino acids have been modified by a deletion, an insertion and/or a substitution, wherein the polypeptide has ability of improving production of monoterpene and/or sesquiterpene in an orchid; and
(c) a polypeptide comprising an amino acid sequence with at least 85% similarity to SEQ ID NO: 2, wherein the polypeptide has ability of improving production of monoterpene and/or sesquiterpene in an orchid.

16. The method according to claim 15, wherein increasing the expression of the protein is by increasing the ploid of a nucleic acid molecule encoding the protein in at least one cell of the orchid.

17. A method for improving production of aroma in an orchid, which comprises transforming the orchid with a vector comprising an isolated nucleic acid molecule, which nucleic acid molecule is selected from the group consisting of:
(a) a nucleic acid molecule, PbbHLH4, comprising a nucleotide sequence of SEQ ID NO: 1;
(b) one or more nucleic acid molecules comprising degenerate sequences of the nucleotide sequences of SEQ ID NO: 1;
(c) a nucleic acid molecule encoding a polypeptide comprising an amino acid sequences of SEQ ID NO: 2;
(d) a nucleic acid molecule encoding a polypeptide comprising an amino acid sequences of SEQ ID NO: 2 with one or more of the amino acids have been modified by a deletion, an insertion and/or a substitution, wherein the polypeptide has ability of improving production of monoterpene and/or sesquiterpene in an orchid;
(e) a nucleic acid molecule encoding a polypeptide comprising an amino acid sequence with at least 85% similarity to SEQ ID NO: 2, wherein the polypeptide has ability of improving production of monoterpene and/or sesquiterpene in an orchid; and
(f) a nucleic acid molecule hybridizes under stringent hybridization conditions to any one of the nucleic acid molecules as defined in (a) to (e).

18. The method according to claim 17, wherein the vector is a shuttle vector that is capable of expressing the nucleic acid molecule in the orchid.

19. The method according to claim 17, wherein the vector comprises an inducible promoter.

20. The method according to claim 17, which comprises transforming the orchid with the vector contained in Agrobacterium tumefaciens.
